(19) United States
(12) Patent Application Publication
(10) Pub. No.: US 2006/0276513 A1
(43) Pub. Date: Dec. 7, 2006

(54) Polymorphs of 3-[(2-{[4-(hexyloxycarbonylamino-imino-methyl)-phenylamino]-methyl}-1-methyl-1H- benzimidazole-5-carbonyl)-pyridin-2-yl-amino]-propionic acid ethyl ester

(75) Inventors: Norbert HAUEL, Schemmerhofen (DE); Rolf SCHMID, Baltringen (DE); Peter SIEGER, Mittelbiberach (DE); Rainer SOBOTTA, Ingelheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/421,153

(22) Filed: May 31, 2006

(30) Foreign Application Priority Data
Jun. 4, 2005 (DE) 10 2005 025 728

(51) Int. Cl. A61K 31/4439; C07D 403/02

(52) U.S. Cl. 514/338; 546/273.4

(57) ABSTRACT

Disclosed are polymorphs of the active substance 3-[(2-{[4-(hexyloxycarbonylamino-imino-methyl)-phenylamino]-methyl}-1-methyl-1H-benzimidazole-5-carbonyl)-pyridin-2 -yl-amino]-propionic acid ethyl ester, the preparation thereof and the use thereof as pharmaceutical compositions.

### APPLICATION DATA

This application claims benefit to DE 10 2005 025 728.3 filed Jun. 4, 2005.

### BACKGROUND OF THE INVENTION

The invention relates to new polymorphs of the active substance 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester, processes for the preparation thereof and the use thereof as pharmaceutical compositions. This active substance with the chemical formula 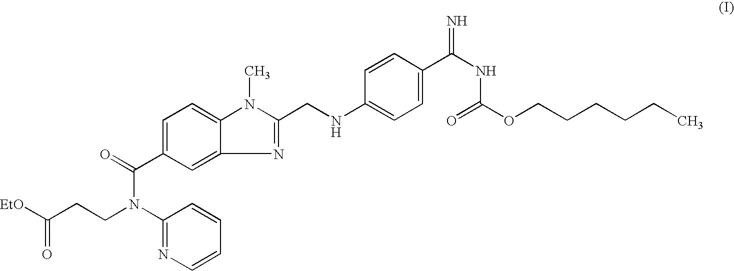
is already known from WO 98/37075, wherein compounds with a thrombin-inhibiting and thrombin time-prolonging activity are disclosed, under the name 1-methyl-2-[N-[4-(N-n-hexyloxycarbonylamidino)phenyl]-amino-methyl]-benzimidazol-5-yl-carboxylic acid-N-(2-pyridyl)-N-(2-ethoxycarbonylethyl)-amide. The compound of formula I is a double prodrug of the compound 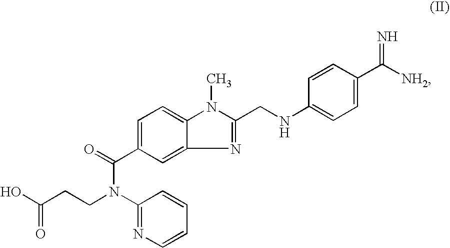
i.e. the compound of formula I is only converted into the actual effective compound, namely the compound of formula II, in the body. The main fields of application of the compound of chemical formula I are the post-operative prophylaxis of deep vein thrombosis and the prevention of stroke.

The aim of the invention is to provide new polymorphs of the compound of formula I having advantageous properties for pharmaceutical use.

The above-mentioned pharmacologically beneficial properties of the disubstituted bicyclic heterocycles disclosed in the prior art are the main prerequisite for effective use of the compounds as pharmaceutical compositions. An active substance must, however, also meet other requirements in order to be capable of being used as a pharmaceutical composition. These parameters are to a large extent connected with the physicochemical nature of the active substance.

Without being restricted thereto, examples of these parameters are the stability of effect of the starting substance under different ambient conditions, stability in the course of the preparation of the pharmaceutical formulation and stability in the final compositions of the pharmaceutical preparation. The pharmaceutical active substance used to prepare the pharmaceutical compositions should therefore have high stability, which should also be guaranteed even under different environmental conditions. This is absolutely essential to prevent the use of pharmaceutical compositions which contain, in addition to the active substance itself, breakdown products thereof, for example. In such cases the content of active substance found in the pharmaceutical formulations might be less than specified.

The absorption of moisture reduces the content of pharmaceutically active substance as a result of the increased weight caused by the uptake of water. Pharmaceutical compositions with a tendency to absorb moisture have to be protected from moisture during storage, e.g. by the addition of suitable drying agents or by storing the drug in an environment where it is protected from moisture. In addition, the uptake of moisture may reduce the content of pharmaceutically active substance during manufacture if the pharmaceutical substance is exposed to the environment without being protected from moisture in any way. Preferably, therefore, a pharmaceutically active substance should be only slightly hygroscopic.

As the crystal modification of an active substance is important to the reproducible active substance content of a preparation, there is a need to clarify as far as possible any existing polymorphism of an active substance present in crystalline form. If there are different polymorphic modifications of an active substance, care must be taken to ensure that the crystalline modification of the substance does not change in the pharmaceutical preparation later produced from it. Otherwise, this could have a harmful effect on the reproducible potency of the drug.

Another criterion which may be of exceptional importance under certain circumstances depending on the choice of formulation or the choice of manufacturing process is the solubility of the active substance. If for example pharmaceutical solutions are prepared (e.g. for infusions), it is essential that the active substance should be sufficiently soluble in physiologically acceptable solvents. It is also very important for drugs which are to be taken orally that the active substance should be sufficiently soluble.

The problem of the present invention is to provide a pharmaceutically active substance which not only is characterised by high pharmacological potency but also satisfies the above-mentioned physicochemical requirements as far as possible.

### DETAILED DESCRIPTION OF THE INVENTION

Surprisingly it has now been found that the novel polymorphs of the compound of formula I (dabigatran etexilate) meet these requirements and have advantageous properties.

The invention therefore relates to the polymorphs of 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester referred to as anhydrous form I, anhydrous form II and tetrahydrate. The invention also relates to pharmaceutical compositions containing at least one of the above-mentioned polymorphs as well as processes for preparing pharmaceutical compositions which are suitable for the prevention of venous thrombosis and stroke and which contain the polymorphs according to the invention.

In a first aspect the present invention therefore relates to the three above-mentioned polymorphic forms of the active substance 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester, preferably in crystalline form, characterised by melting points of T_{mp.}=135±3° C. (anhydrous form I), T_{mp.}=150±3° C. (anhydrous form II) or T_{mp.}=90±5° C. (tetrahydrate) (determined by DSC; evaluation by peak maximum; heating rate: 10° C./min). The DSC diagram of the anhydrous form I is characterised in that four other weakly endothermic signals can be observed at about 53, 75, 98 and 118° C. These signals can be attributed to fully reversible solid-to-solid phase transitions, i.e. in the temperature range between 53-75, 75-98, 98-118 and 118-135° C. there are four other high temperature phases of the anhydrous form I.

The invention also relates to the methods of selectively producing the three polymorphic forms as well as the modifications which may be obtained by these methods.

According to the invention the anhydrous form I of 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester is obtained by
a) dissolving 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester base in ethyl acetate at reflux temperature,
b) cooling the solution to a temperature of about 30° C. to 35° C. and stirring for a further 60 minutes at this temperature,
c) cooling to about 15° C. to 20° C. and stirring for about another 60 minutes at this temperature,
d) suction filtering the precipitated crystals, washing them with ethyl acetate and
e) drying the product thus obtained at 40 to 50° C. in a circulating air dryer.

According to the invention the anhydrous form II of 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester is obtained by
a) combining 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester base with a little ethyl acetate,
b) heating the resulting suspension to a temperature of about 80° C., to obtain a clear solution,
c) refluxing the solution for about one hour,
d) filtering off the precipitated crystals and
e) drying the product thus obtained in the air.

According to the invention the tetrahydrate of 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester is obtained by
a) dissolving 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester base in acetone/water (80:20) with shaking, at a temperature of about 60° C.,
b) cooling the solution to a temperature of about 30° C. and filtering it into a sealable vessel,
c) cooling the sealed vessel containing the solution to a temperature of about −9° C. and leaving it for about 30 minutes at this temperature,
d) adding a mixture of acetone and water (80:20) which has been pre-cooled to −9° C. and shaking the mixture,
e) suction filtering the precipitated crystals and washing them with a mixture of acetone and water (80:20) which has been cooled to −9° C., and
f) drying the product thus obtained in the air at ambient temperature.

The crystalline forms of 3-[(2-{[4-(hexyloxycarbonylamino-imino-methyl)-phenyl-amino]-methyl}-1-methyl-1H-benzimidazole-5-carbonyl)-pyridin-2-yl-amino]-propionic acid ethyl ester according to the invention were investigated in more detail by x-ray powder diffraction. The diagrams obtained are shown in FIGS. 1 to 3.

TABLE 1
X-ray powder reflections (up to 30° 2 Θ) and intensities (standardised)
of 3-[(2-{[4-(hexyloxycarbonylamino-imino-methyl)-phenylamino]-
methyl}-1-methyl-1H-benzimidazole-5-carbonyl)-pyridin-2-yl-amino]-
propionic acid ethyl ester (anhydrous form I)

| 2 Θ [°] | d [Å] | I/I₀ [%] |
|---|---|---|
| 3.39 | 26.07 | 1 |
| 4.31 | 20.51 | 1 |
| 4.87 | 18.12 | 10 |
| 5.62 | 15.70 | 1 |
| 7.28 | 12.14 | 1 |
| 7.43 | 11.88 | 1 |
| 8.82 | 10.02 | 69 |
| 10.46 | 8.45 | 100 |
| 11.56 | 7.65 | 25 |
| 12.92 | 6.84 | 11 |
| 13.25 | 6.67 | 32 |
| 13.78 | 6.42 | 50 |
| 14.12 | 6.27 | 12 |
| 14.36 | 6.16 | 9 |
| 14.67 | 6.03 | 5 |
| 15.49 | 5.72 | 63 |
| 16.61 | 5.33 | 28 |
| 17.76 | 4.99 | 4 |
| 18.03 | 4.92 | 12 |
| 18.93 | 4.69 | 4 |
| 20.12 | 4.41 | 65 |
| 20.79 | 4.27 | 6 |
| 21.54 | 4.12 | 46 |
| 22.15 | 4.01 | 15 |
| 22.72 | 3.91 | 6 |
| 23.28 | 3.82 | 11 |
| 23.51 | 3.78 | 11 |
| 23.92 | 3.72 | 18 |
| 24.18 | 3.68 | 8 |
| 24.58 | 3.62 | 10 |
| 25.18 | 3.53 | 8 |
| 25.70 | 3.46 | 69 |
| 26.75 | 3.33 | 10 |
| 27.69 | 3.22 | 21 |
| 28.27 | 3.15 | 14 |
| 28.80 | 3.10 | 9 |
| 29.41 | 3.03 | 2 |
| 30.16 | 2.96 | 2 |

TABLE 2
X-ray powder reflections (up to 30° 2 Θ) and intensities (standardised)
of 3-[(2-{[4-(hexyloxycarbonylamino-imino-methyl)-phenylamino]-
methyl}-1-methyl-1H-benzimidazole-5-carbonyl)-pyridin-2-yl-amino]-
propionic acid ethyl ester (anhydrous form II)

| 2 Θ [°] | d [Å] | I/I₀ [%] |
|---|---|---|
| 7.55 | 11.70 | 6 |
| 8.25 | 10.71 | 24 |
| 8.50 | 10.40 | 18 |
| 9.06 | 9.75 | 44 |
| 10.04 | 8.80 | 86 |
| 11.08 | 7.98 | 5 |
| 11.99 | 7.37 | 33 |
| 12.17 | 7.27 | 51 |
| 13.11 | 6.75 | 4 |
| 13.77 | 6.42 | 19 |
| 14.04 | 6.30 | 5 |
| 14.67 | 6.04 | 3 |
| 14.98 | 5.91 | 13 |
| 15.10 | 5.86 | 11 |
| 15.93 | 5.56 | 3 |
| 16.34 | 5.42 | 20 |
| 16.52 | 5.36 | 55 |
| 17.03 | 5.20 | 68 |
| 17.63 | 5.03 | 47 |
| 17.99 | 4.93 | 7 |
| 18.17 | 4.88 | 5 |
| 18.74 | 4.73 | 6 |
| 19.00 | 4.67 | 19 |
| 19.23 | 4.61 | 6 |
| 19.69 | 4.51 | 17 |
| 20.02 | 4.43 | 53 |
| 20.43 | 4.34 | 11 |
| 20.63 | 4.30 | 48 |
| 20.92 | 4.24 | 3 |
| 21.24 | 4.18 | 7 |
| 21.39 | 4.15 | 10 |
| 21.75 | 4.08 | 4 |
| 22.15 | 4.01 | 9 |
| 22.41 | 3.96 | 6 |
| 22.77 | 3.90 | 17 |
| 23.02 | 3.86 | 6 |
| 23.17 | 3.84 | 10 |
| 23.70 | 3.75 | 10 |
| 23.92 | 3.72 | 19 |
| 24.49 | 3.63 | 13 |
| 24.69 | 3.60 | 15 |
| 24.89 | 3.57 | 12 |
| 25.17 | 3.53 | 26 |
| 25.66 | 3.47 | 6 |
| 25.88 | 3.44 | 10 |
| 26.36 | 3.38 | 100 |
| 26.67 | 3.34 | 21 |
| 26.85 | 3.32 | 15 |
| 27.96 | 3.19 | 7 |
| 28.24 | 3.16 | 7 |
| 28.52 | 3.13 | 5 |
| 28.79 | 3.10 | 9 |
| 29.81 | 2.99 | 5 |
| 30.11 | 2.97 | 9 |

TABLE 3
X-ray powder reflections (up to 30° 2 Θ) and intensities (standardised)
of 3-[(2-{[4-(hexyloxycarbonylamino-imino-methyl)-phenylamino]-
methyl}-1-methyl-1H-benzimidazole-5-carbonyl)-pyridin-2-yl-amino]-
propionic acid ethyl ester (tetrahydrate)

| 2 Θ [°] | d [Å] | I/I₀ [%] |
|---|---|---|
| 4.67 | 18.91 | 100 |
| 6.63 | 13.32 | 2 |
| 8.10 | 10.90 | 45 |
| 9.36 | 9.44 | 20 |
| 9.82 | 9.00 | 52 |
| 10.55 | 8.38 | 2 |
| 11.18 | 7.91 | 32 |
| 11.91 | 7.42 | 2 |
| 12.57 | 7.04 | 10 |
| 13.01 | 6.80 | 9 |
| 13.52 | 6.54 | 5 |
| 14.05 | 6.30 | 23 |
| 14.59 | 6.06 | 18 |
| 15.52 | 5.70 | 8 |
| 15.99 | 5.54 | 10 |
| 16.31 | 5.43 | 8 |
| 16.56 | 5.35 | 14 |
| 16.94 | 5.23 | 21 |
| 17.76 | 4.99 | 16 |
| 18.62 | 4.76 | 33 |
| 18.93 | 4.68 | 26 |
| 19.69 | 4.51 | 10 |
| 20.10 | 4.42 | 15 |
| 20.56 | 4.32 | 52 |
| 20.86 | 4.26 | 7 |
| 21.38 | 4.15 | 19 |
| 21.83 | 4.07 | 24 |
| 22.51 | 3.95 | 22 |
| 23.10 | 3.85 | 34 |
| 24.04 | 3.70 | 33 |
| 24.74 | 3.60 | 83 |
| 25.23 | 3.53 | 8 |
| 25.58 | 3.48 | 12 |
| 26.35 | 3.38 | 9 |
| 26.96 | 3.30 | 21 |
| 28.31 | 3.15 | 9 |
| 28.87 | 3.09 | 2 |
| 29.32 | 3.04 | 5 |
| 23.92 | 3.72 | 19 |
| 24.49 | 3.63 | 13 |
| 24.69 | 3.60 | 15 |
| 24.89 | 3.57 | 12 |
| 25.17 | 3.53 | 26 |
| 25.66 | 3.47 | 6 |
| 25.88 | 3.44 | 10 |
| 26.36 | 3.38 | 100 |
| 26.67 | 3.34 | 21 |
| 26.85 | 3.32 | 15 |
| 27.96 | 3.19 | 7 |
| 28.24 | 3.16 | 7 |
| 28.52 | 3.13 | 5 |
| 28.79 | 3.10 | 9 |
| 29.81 | 2.99 | 5 |
| 30.11 | 2.97 | 9 |

In the preceding Tables 1 to 3 the value “2 Θ [°]” denotes the angle of diffraction in degrees and the value “d [Å]” denotes the distances in Å determined between the lattice planes.

The x-ray powder diagrams were recorded, within the scope of the present invention, using a STOE STADI P diffractometer fitted with a location-sensitive detector (OED) and a Cu anode as the x-ray source and a primary monochromator (CuK_{α1} radiation, X=1.54056 Å, 40 kV, 40 mA).

### BRIEF DESCRIPTION OF THE FIGURES

FIGS. 1 to 3 show the X-ray powder diffractograms of the three crystalline forms of 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester.

FIGS. 4 to 6 show the thermoanalysis (DSC/TG) for the three crystalline forms of 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester.

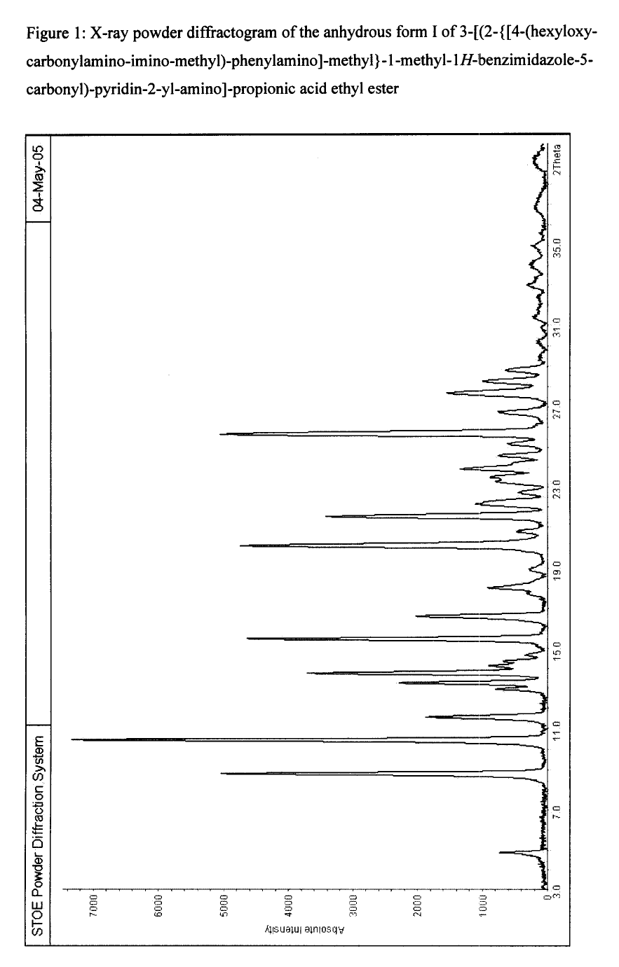

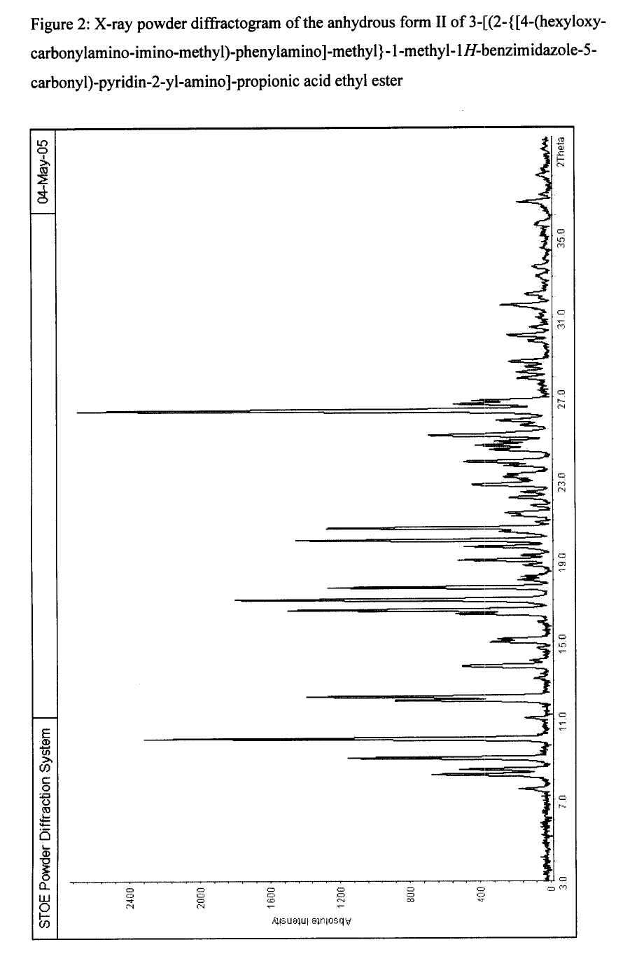

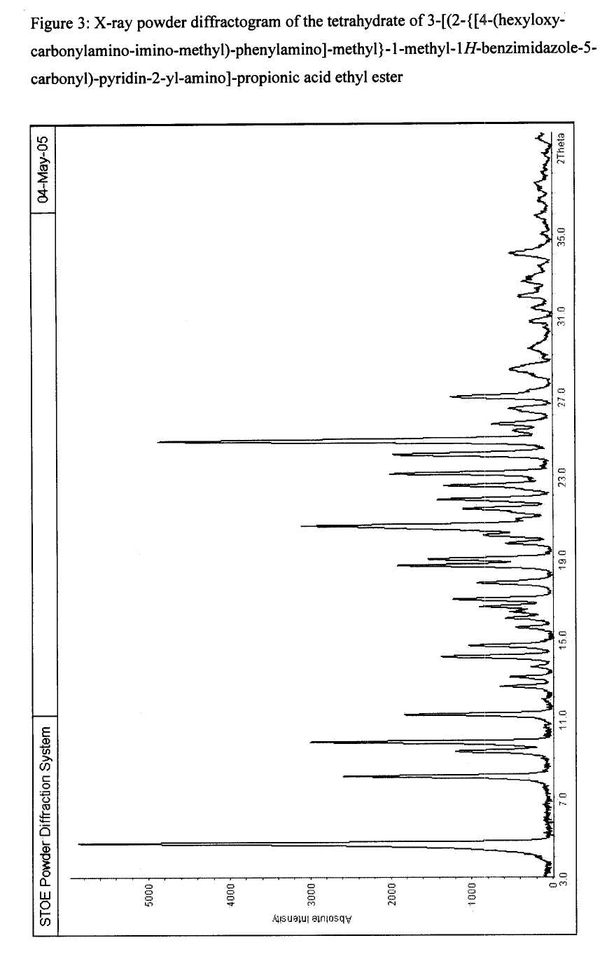

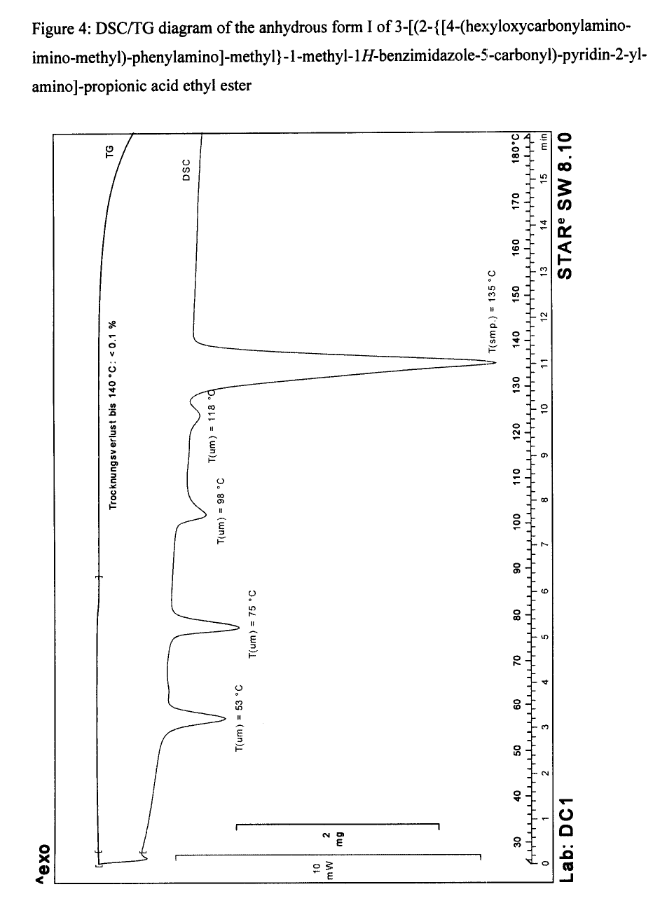

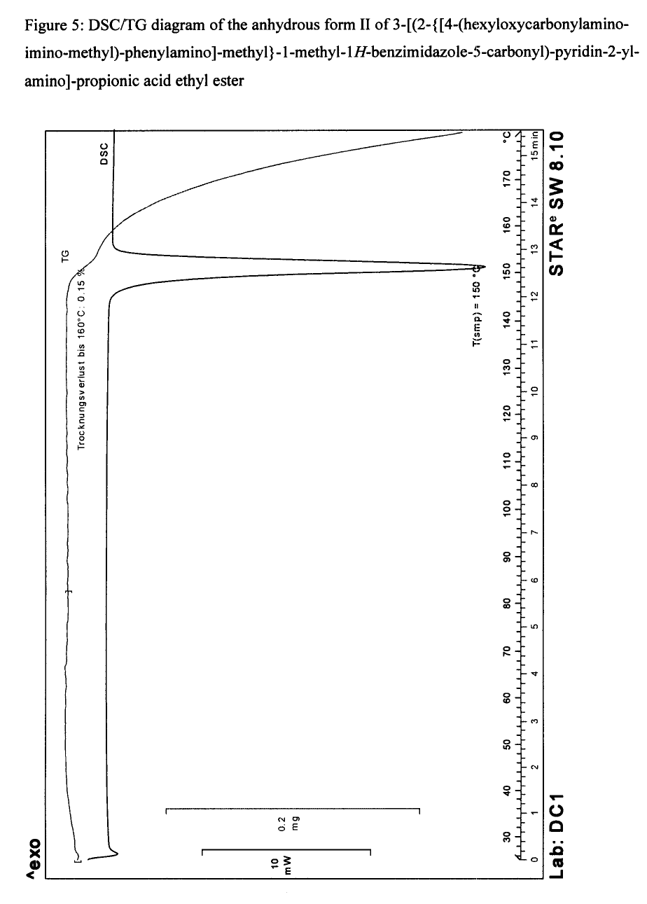

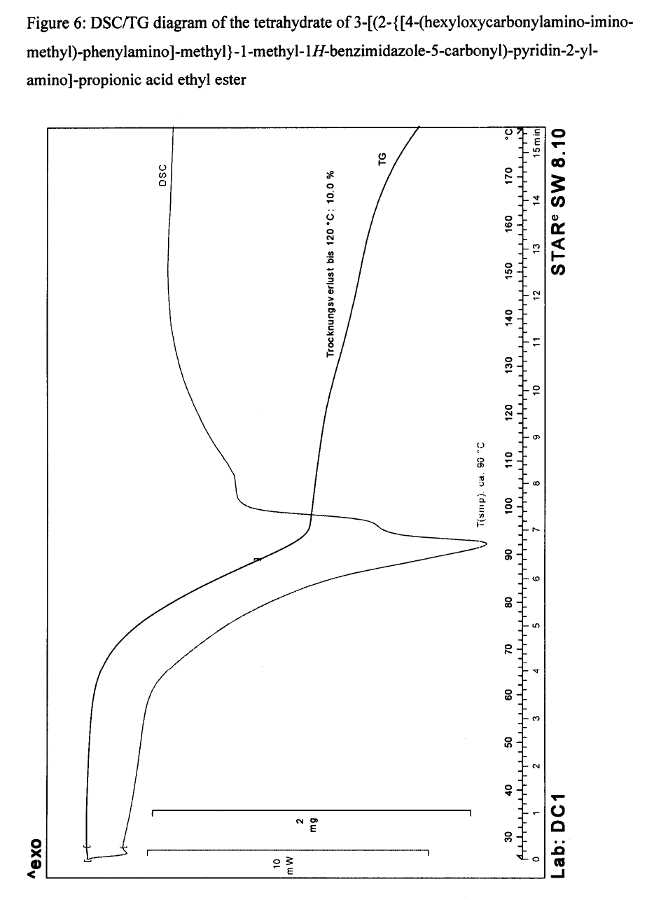

Captions to FIGS. 4 to 6: Trocknungsverlust bis=drying loss up to T(smp.)=T(mp.)

### EXAMPLES

The melting points were determined by DSC, using an apparatus obtained from Mettler-Toledo (type: DSC 821). The melting temperature used was the peak temperature of the corresponding melting peak in the DSC diagram. The accuracy of the melting points specified is about ±3° C., or ±5° C. in the case of the tetrahydrate, as the tetrahydrate, as it melts, releases the crystal water locked in the crystal lattice and produces a greatly propagated endothermic signal.

The starting compound 3-[(2-{[4-(amino-hexyloxycarbonylimino-methyl)-phenylamino]-methyl}-1-methyl-]H-benzimidazole-5-carbonyl)-pyridin-2-yl-amino]-propionic acid ethyl ester may for example be prepared as described in International Application WO 98/37075, Example 113.

### Example 1

### 3-[(2-{[4-(amino-hexyloxycarbonylimino-methyl)-phenylamino]-methyl}-1-methyl-1H-benzimidazole-5-carbonyl)-pyridin-2-yl-amino]-propionic acid ethyl ester (anhydrous form I)

1500 g (2.389 mol) 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester base (prepared as described in WO 98/37075) are dissolved in 12 litres of ethyl acetate at reflux temperature. The solution is cooled to 30-35° C. After a few minutes the product began to crystallise out. It was stirred for another 60 minutes at 30-35° C. and for another 60 minutes at 15-20° C., then the precipitate was suction filtered, washed with 3 litres of ethyl acetate and dried at 40-50° C. in the circulating air dryer.

Yield: 88.5 % of theory

### Example 2

### 3-[(2-{[4-(amino-hexyloxycarbonylimino-methyl)-phenylamino]-methyl}-1-methyl-1H-benzimidazole-5-carbonyl)-pyridin-2-yl-amino]-propionic acid ethyl ester (anhydrous form II)

2.0 g of 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester base (prepared as described in WO 98/37075) were combined with 10 ml of ethyl acetate. The resulting suspension was heated to 80° C. (substance goes into solution) and refluxed for 1 hour. After about 30 minutes at reflux temperature form II begins to crystallise. Then the precipitated substance was filtered off and dried in the air.

Yield: 85 % of theory

### Example 3

### 3-[(2-{[4-(amino-hexyloxycarbonylimino-methyl)-phenylamino]-methyl}-1-methyl-1H-benzimidazole-5-carbonyl)-pyridin-2-yl-amino]-propionic acid ethyl ester tetrahydrate

0.5 g of 3-[(2-{[4-(hexyloxycarbonylamino-imino-methyl)-phenylamino]-methyl}-1-1H-benzimidazole-5-carbonyl)-pyridin-2-yl-amino]-propionic acid ethyl ester base (prepared as described in WO 98/37075) are dissolved in 5 ml of a mixture of acetone/water=80:20 at 60° C. with agitation. The solution was cooled to about 30° C. and filtered through a filter, for example through a Sartorius Minisart Filter SRP 15 into a 10 ml glass vial, and the flask was sealed. The solution was then cooled in an ice/ethanol mixture to about −9° C. The substance began to crystallise out by itself. After about 30 minutes in the ice bath, about 3 ml of a mixture of acetone and water (80:20) cooled to −9° C. were added, the mixture was agitated and then suction filtered through a filter, for example a Schleicher & Schiill round filter no. 595.

It was rinsed with approximately another 5 ml of a mixture of acetone and water (80:20) cooled to −9° C. The substance filtered off was scraped off the round filter into a crystallising dish and dried in the air at ambient temperature.

Yield: 97 % of theory

### Example 4

Dry Ampoule Containing 75 mg of Active Substance Per 10 ml

Composition:

| | | |
|---|---|---|
| Active substance | 75.0 | mg |
| Mannitol | 50.0 | mg |
| water for injections | ad 10.0 | ml |

Preparation:

Active substance and mannitol are dissolved in water. After packaging the solution is freeze-dried. To produce the solution ready for use, the product is dissolved in water for injections.

### Example 5

Dry Ampoule Containing 35 mg of Active Substance Per 2 ml

Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging, the solution is freeze-dried.

To produce the solution ready for use, the product is dissolved in water for injections.

### Example 6

Tablet Containing 50 mg of Active Substance

Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 9 mm.

### Example 7

Tablet Containing 350 mg of Active Substance

Composition:

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 12 mm.

### Example 8

Capsules Containing 50 mg of Active Substance

Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation:

(1) is triturated with (3). This trituration is added to the mixture of (2) and (4) with vigorous mixing.

This powder mixture is packed into size 3 hard gelatine capsules in a capsule filling machine.

### Example 9

Capsules Containing 350 mg of Active Substance

Composition:

| | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

Preparation:

(1) is triturated with (3). This trituration is added to the mixture of (2) and (4) with vigorous mixing.

This powder mixture is packed into size 0 hard gelatine capsules in a capsule filling machine.

### Example 10

Suppositories Containing 100 mg of Active Substance

1 suppository contains:

| | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

### Example 11

| | percentage composition | | | | | |
|---|---|---|---|---|---|---|
| | | | active | | per | per |
| | core | insulating | substance | | capsule | capsule |
| | material | layer | layer | total | [mg] | [mg] |
| tartaric acid | 61.3 | — | — | 61.3 | 176.7 | 353.4 |
| gum arabic | 3.1 | 2.8 | | 5.9 | 17.0 | 34.0 |
| talc | — | 5.6 | 3.2 | 8.8 | 25.4 | 50.7 |
| hydroxypropylcellulose | — | — | 4.0 | 4.0 | 11.5 | 23.1 |
| active substance (in | — | — | 20.0 | 20.0 | 50.0 | 100.0 |
| relation to the base) | | | | | | |
| total | | | | 100.0 | 288.3 | 576.5 |

### Example 12

| | percentage composition | | | | | |
|---|---|---|---|---|---|---|
| | | | active | | per | per |
| | core | insulating | substance | | capsule | capsule |
| | material | layer | layer | total | [mg] | [mg] |
| tartaric acid | 38.5 | — | — | 38.5 | 55.5 | 166.5 |
| gum arabic | 1.9 | 1.7 | | 3.6 | 5.2 | 15.6 |
| talc | — | 3.5 | 6.4 | 9.9 | 14.3 | 42.8 |
| hydroxypropylcellulose | — | — | 8.0 | 8.0 | 11.5 | 34.6 |
| active substance (in | — | — | 40.0 | 40.0 | 50.0 | 150.0 |
| relation to the base) | | | | | | |
| total | | | | 100.0 | 144.2 | 432.5 |

The preparation and structure of the pellets according to Example 11 and 12 are described in detail in WO 03/074056.

1. A compound which is 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester in crystalline form, wherein the compound has a melting point of T_{mp.}=135±3° C. (anhydrous form I).

2. A compound which is 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester in crystalline form, wherein the compound has a melting point of T_{mp.}=150±3° C. (anhydrous form II).

3. A compound which is 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester tetrahydrate in crystalline form, wherein the compound has a melting point of T_{mp.}=90±5° C. with simultaneous release of the crystal water locked into the crystal lattice (tetrahydrate).

4. A method for the prevention of venous thromboses and stroke comprising administering to a patient a pharmaceutically effective amount of a compound according to one of claims 1 to 3.

5. A pharmaceutical composition comprising a pharmaceutically effective amount of a compound according to one of claims 1 to 3, optionally together with one or more inert carriers and/or diluents.

6. A process for preparing anhydrous form I of 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester, comprising
a) dissolving 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester base in ethyl acetate at reflux temperature,
b) cooling the solution to a temperature of about 30° C. to 35° C. and stirring for another 60 minutes at this temperature,
c) cooling to about 15° C. to 20° C. and stirring for about another 60 minutes at this temperature,
d) filtering the precipitated crystals via suction, washing with ethyl acetate and
e) obtaining the product dried at 40 to 50° C. in the circulating air dryer.

7. A process for preparing anhydrous form II of 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester, comprising
a) combining 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester base with a small amount ethyl acetate,
b) heating the suspension obtained to a temperature of about 80° C., whereupon a clear solution is formed,
c) refluxing the solution for about one hour,
d) filtering the crystals precipitated and
e) obtaining the product dried in the air.

8. A process for preparing the tetrahydrate of 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester, comprising
a) dissolving 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester base in acetone/water (80:20) and agitating at a temperature of about 60° C.,
b) cooling the solution to a temperature of about 30° C. and filtering into a sealable vessel,
c) cooling the sealed vessel containing the solution to a temperature of about −9° C. and leaving the sealed vessel containing the solution for about 30 minutes at this temperature,
d) adding a mixture of acetone and water (80:20) pre-cooled to −9° C. and agitating the mixture,
e) filtering the crystals precipitated via suction and washing with a mixture of acetone and water (80:20) cooled to −9° C. and
f) obtaining the product dried in the air at ambient temperature.

9. A product 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester, anhydrous form I, obtainable by the process according to claim 6.

10. A product 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester, anhydrous form II, obtainable by the process according to claim 7.

11. A product 3-[(2-{[4-(hexyloxycarbonylamino-imino-methyl)-phenylamino]-methyl}-1-methyl-1H-benzimidazole-5-carbonyl)-pyridin-2-yl-amino]-propionic acid ethyl ester tetrahydrate, obtainable by the process according to claim 8.
